(19) United States
(12) Patent Application Publication
(10) Pub. No.: US 2011/0054629 A1
(43) Pub. Date: Mar. 3, 2011

(54) COMPOSITE IMPLANT HAVING POROUS STRUCTURE FILLED WITH BIODEGRADABLE ALLOY AND METHOD OF MAGNESIUM-BASED MANUFACTURING THE SAME

(75) Inventors: Hyun-Kwang Seok, Seoul (KR); Seok-Jo Yang, Daejeon (KR); Yu-Chan Kim, Seoul (KR); Ja-Kyo Koo, Seoul (KR)

(73) Assignee: U&I CORPORATION, Gyeonggi-do (KR)

(21) Appl. No.: 12/933,462

(22) PCT Filed: Mar. 18, 2009
(86) PCT No.: PCT/KR2009/001361

§ 371 Date: Sep. 20, 2010

(30) Foreign Application Priority Data
Mar. 18, 2008 (KR) 10-2008-0024801

(51) Int. Cl. A61F 2/28; B05D 7/00; B05D 3/02

(52) U.S. Cl. 623/23.6; 427/2.26

(57) ABSTRACT

The present invention provides a composite implant comprising pores of a porous structure filled with a biodegradable magnesium-based alloy. Further, the present invention provides a composite implant which filles pores of the porous structure prepared by a metal, a ceramic or a polymer with a biodegradable magnesium-based alloy. Mechanical properties of the composite implant of the present invention are improved because a magnesium-based alloy filled in its pores increases the strength of a porous structure comprised of a metal, a ceramic or a polymer. Further, it can be expected that the magnesium-based alloy filled in the porous structure is decomposed in a living body, thus increasing bone formation rate. Accordingly bone tissue can be rapidly formed because the composite implant of the present invention has high strength and excellent interfacial force between the composite implant and bone tissue, compared to conventional porous materials.

### TECHNICAL FIELD

The present invention relates to a composite implant having a porous structure filled with a biodegradable alloy and a method of manufacturing the same. More specifically, the present invention relates to a composite implant having a porous structure filled with magnesium or magnesium-based alloy which is biodegradable and thus has a controllable biodegradation rate, which has high strength and excellent interfacial force between the magnesium or magnesium-based alloy and bone tissue and which can improve a bone formation rate, and a method of manufacturing the same.

### BACKGROUND ART

Typical materials of implants used for medical treatment include metals, ceramics, polymerics and the like. Among them, metallic implants have excellent mechanical properties and workability, but have disadvantages such as stress shielding, image degradation, implant migration and the like. Further, ceramic implants have relatively excellent biocompatibility, but are disadvantageous in that they are easily damaged by external impacts and are difficult to manufacture. Further, polymeric implants are disadvantageous in that they have relatively low strength compared to other implant materials.

Meanwhile, recently, porous implants which can accelerate the formation of bone tissue when they are introduced into a human body and which can prevent a stress shielding phenomenon by decreasing Young's modulus have been developed. However, these porous implants are disadvantageous in that they are vulnerable to external impacts due to their low mechanical strengths.

Further, research and development into biodegradable implants which are not required to be removed from a human body after they are introduced into the human body as a surgical operation and desired results are obtained has been conducted. The medical application of biodegradable materials began to have been researched since the middle stage of the 1960's, based on polymers such as polylactic acid (PLA), polyglycolic acid (PGA) and copolymers thereof (PLGA) and the like. However, such biodegradable polymers are problematic in that they have low mechanical strengths, in that acids are formed when they are decomposed and in that it is difficult to control their biodegradation rates, and thus the application thereof has been limited. In particular, it was very difficult to apply biodegradable polymers to orthopedic or dental implants which are subjected to a heavy load due to low mechanical strengths.

Therefore, in order to overcome the disadvantages of biodegradable polymers, research into several biodegradable materials has been conducted. Typical examples of the biodegradable materials may include ceramics such as tri-calcium phosphate (TCP) and the like, and composite materials of biodegradable polymers and biodegradable hydroxyapatite (HA). However, the mechanical properties of these materials are not remarkably different from those of biodegradable polymers. Particularly, the ceramic materials have fatal disadvantages as biomaterials because of their poor impact resistances. Further, there is some doubt whether these materials prove practically effective because it is difficult to control their biodegradation rate.

### DISCLOSURE OF INVENTION

### Technical Problem

Accordingly, the present invention has been made to solve the above-mentioned conventional problems, and an object of the present invention is to provide a composite implant which can make up for the disadvantages, such as low mechanical strength and poor impact resistance, of conventional porous implants.

Another object of the present invention is to provide a composite implant which can increase bone formation rate and which can be replaced with bone tissue because biodegradable metals filled in its pores are removed when the passage of a predetermined time after the introduction into a human body.

A further object of the present invention is to provide a composite implant which improves both the corrosion resistance and mechanical properties thereof by adjusting the amount of impurities in a magnesium-based alloy.

### Technical Solution

In order to accomplish the above objects, the present invention provides a composite implant having a porous structure whose pores are filled with a biodegradable magnesium-based alloy.

Further, the present invention provides a method of manufacturing a composite implant, including the steps of: a) preparing a porous structure; and b) filling pores of the porous structure with a biodegradable magnesium-based alloy to form a composite material.

### ADVANTAGEOUS EFFECTS

According to the biodegradable composite implant of the present invention, bone formation rate is increased by the formation of blood vessels through pores. Specifically, when the biodegradable composite implant according to the present invention was introduced into the human body, a magnesium-based alloy filled in the inner pores decomposes as time passes, and the pores of the porous structure become vacant, so that blood vessels are formed through the vacant pores of the porous structure, thereby accelerating the formation of bone.

Further, according to the biodegradable composite implant of the present invention, a stress shielding phenomenon is prevented due to the decrease of Young's modulus. Specifically, the inner parts of pores are filled with magnesium (Mg) having a low Young's modulus, so that the Young's modulus of the biodegradable composite implant according to the present invention also becomes low, thereby preventing the stress shielding phenomenon.

Further, according to the biodegradable composite implant of the present invention, low strength and impact resistance, which are fatal disadvantages of conventional porous implants, can be improved. Additionally, bone formation rate can be further improved because a biodegradable magnesium-based alloy filled in its pores slowly dissolves after it is introduced into a human body, thus promoting a bone formation reaction, that is, a hydroxyapatite (HA) formation reaction.

Further, according to the biodegradable composite implant of the present invention, the porosity of a porous structure and the composition of an impregnated magnesium-based alloy can be changed, thus controlling the strength of the biodegradable composite implant, the decomposition rate and bone formation rate of the impregnated metal alloy.

Due to the above-mentioned advantages, the biodegradable composite implant according to the present invention is suitable for a bone, substitute or treatment for bone and the like, and can be used as an orthopedic implant, a dental implant, an implant for a plastic surgery or an implant for blood vessels.

### BRIEF DESCRIPTION OF DRAWINGS

FIG. 1 is a photograph showing the outer appearance of an alumina porous structure;

FIG. 2 is a photograph showing the outer appearance of an alumina implant whose pores are impregnated with magnesium (Mg);

FIG. 3 is a photograph showing the section of an alumina implant whose pores are filled with magnesium (Mg);

FIG. 4 is a photograph showing the outer appearance of a titanium porous structure formed by sintering titanium (Ti) powder;

FIG. 5 is a photograph showing the section of a titanium porous implant material whose pores are filled with magnesium (Mg);

FIG. 6 shows a test scene for examining the surface strength of a porous structure and a porous implant whose pores are filled with magnesium (Mg) and a shape of a tip used in the test;

FIG. 7 is a graph showing the change of penetration depth of the surface of an alumina porous structure according to the load applied thereto; and

FIG. 8 is a graph showing the change of penetration depth of the surface of an alumina implant whose pores are filled with magnesium (Mg) according to the load applied thereto.

### BEST MODE FOR CARRYING OUT THE INVENTION

Hereinafter, preferred embodiments of the present invention will be described in detail.

I. Composite Implant

A composite implant of the present invention includes a porous structure whose pores are filled with a magnesium-based alloy. The magnesium-based alloy may include pure magnesium and alloys thereof, and may be represented by Chemical Formula 1 as below:

MgₐCa_{b}X_{c} <Chemical Formula 1>

wherein a, b and c are molar ratios of respective components and are in the range of 0.5≦a≦1, 0≦b≦0.4, and 0≦c≦0.4; and

X includes one or more selected from the group consisting of zirconium (Zr), molybdenum (Mo), niobium (Nb), tantalum (Ta), titanium (Ti), strontium (Sr), chromium (Cr), manganese (Mn), zinc (Zn), silicon (Si), phosphorus (P), nickel (Ni), iron (Fe) and selenium (Se).

Even when X includes two or more selected from the group as magnesium-based alloys, the total molar ratio thereof satisfies 0≦c≦0.4. As the content of Ca and X is increased, the strength of the magnesium-based alloy is increased, and simultaneously biodegradation rate is also increased. Accordingly, in a biodegradable implant, the amount of Ca and X is determined within the above-mentioned range in consideration of the desired strength and the decomposition rate of the filled metals.

When X includes nickel (Ni), nickel reduces toxicity in a living body and control corrosion rate. The content of nickel may be 100 ppm or less, preferably 50 ppm or less. Further, when X includes iron (Fe), iron very greatly influences in the increase of corrosion rate of a magnesium-based alloy, and thus the content of iron may be 1000 ppm or less exceeding 0 ppm, preferably 500 ppm or less. Here, when the content of iron includes more than 1000 ppm, iron exists as an independent factor, not a solid solution, because iron is not engaged in magnesium, thus increasing the corrosion rate of magnesium. Further, while magnesium is decomposed in living bodies, iron, which independently exists in a magnesium-based alloy, may flow into the living bodies.

A porous structure having a pore size of 200˜500 μm is preferable in the composite implant of the present invention. The pore size may be adjusted using a general method known in the related technical field according to the used field. When the pore size meets the above-mentioned range, blood vessels serving to supply nutriments, minerals and ions can easily pass through the pores.

It is preferred that the porous structure have a porosity of 5˜95%. Here, the porosity is a volume ratio of pores to the total volume of the porous structure. When the desired strength of the object to which the porosity is applied is high, the strength of the porous structure can be increased by decreasing the porosity. Specifically, for example, when the porous structure is comprised of tantalum having high strength or serves to fill simply lost cavities of bone, the porosity thereof may be increased.

The porous structure may be formed using one or more selected from the group consisting of metals, ceramics and polymers. When the porous structure is comprised of the metal, the metal may be selected from the group consisting of titanium, titanium alloys, cobalt-chromium alloys and stainless steels. When the porous structure is comprised of a ceramic, the ceramic may be selected from the group consisting of calcium phosphate, alumina, zirconia and magnesia. When the porous structure is comprised of a polymer, the polymer may be selected from the group consisting of polyethylene, polylactic acid (PLA), polyglycolic acid (PGA), and copolymers (PLGA) thereof. Here, when the porous structure is made of the polymer, biodegradable acids are produced, and thus pH is decreased. In this case, since a polymer composite material whose pores are filled with magnesium has an effect in which pH is increased by the decomposition of magnesium, it will be additionally expected that the pH in a living body can be arbitrarily adjusted by controlling the de-composition rate of polymers and magnesium.

The above-mentioned biodegradable composite implant according to the present invention may be used as an orthopedic implant, a dental implant, an implant for a plastic surgery or an implant for a blood vessel. Specifically, the biodegradable composite implant can be used as an implant for an interbody spacer for vertebra, a bone filler, a bone plate, a bone pin, a bone screw, a scaffold, or an artificial dential root and the ike.

II. Method of Manufacturing a Composite Implant

Hereinafter, a method of manufacturing a composite implant according to the present invention will be described.

The method of manufacturing a biodegradable composite implant according to the present invention includes the steps of: a) preparing a porous structure; and b) filling pores of the porous structure with a biodegradable magnesium-based alloy to form a composite material.

In the step a), the porous structure may be made of any one selected from the group consisting of metals, ceramics and polymers.

In the case where the porous structure is prepared using only a metal, the step a) is as follows. First, metal is formed into powder or a wire. The metal powder or metal wire is formed into a green preform. The preform can be formed through a sintering process or a modification thereof.

The method using the sintering process is conducted as follows. First, the metal powder or metal wire is put into a container or is pressurized at a pressure of 100 MPa or less so as to have low strength. The metal powder or metal wires having the low strength are maintained at a temperature of 2/10˜ 9/10 of its melting point, and are thus connected with each other to form a preform having mechanical strength.

Further, the method using the modified sintering process is conducted as follows. First, the metal powder or metal wire is put into a conductive container made of graphite, and then high current is applied to the conductive container to generate heat from the contact portion of the meal powder or metal wire, and thus a sintered body is made, thereby forming a preform.

In the case where the porous structure is prepared including a metal and a polymer, the step a) is as follows. First, metal is formed into powder or a wire. Subsequently, the metal powder or metal wire is mixed with a polymer, so that the polymer decomposes and disappears at low temperature in the process of increasing the temperature and the metal powder or metal wire is sintered at high temperature, thereby forming a preform having appropriate mechanical strength. In this case, the porosity and strength of the preform are determined by sintering temperature, pressure, mixing ratio of metal and polymer and the like, and, if necessary, suitable conditions can be selected. The sintering temperature changes depending on the kinds of the materials used to prepare a porous structure, and may be generally about ½˜ 9/10 of the melting point of the porous structure. The metal powder or metal wire is sintered even when it is not pressurized at sintering, but it is rapidly sintered as the pressure applied thereto is increased. However, as the pressure is higher, additional costs such as equipment costs, mold costs and the like, are required, and thus adequate pressure may be selected.

Alternatively, in the case where the porous structure is prepared including a metal and a polymer, the step a) may be as follows.

First, the surface of the polymer is plated with precious metals such as gold (Au), platinum (Pt), palladium (Pd) or the like. Subsequently a methal porous structure having better biocompatibility can be prepared by removing the polymer.

Meanwhile, in the case where the porous structure is prepared using a water-soluble salt and a metal, the step a) is as follows.

First, a water-soluble salt and metal powder are mixed and then molded at high temperature to form a preform. Here, the water-soluble salt may be one or more selected from the group consisting of NaNO₂, KNO₂, NaNO₃, NaCl, CuCl, KNO₃, KCl, LiCl, KNO₃, PbCl₂, MgCl₂, CaCl₂ and BaCl₂. Subsequently, the preform is pressurized at the temperature of 2/10˜ 9/10 of the melting point of the metal powder. In the process of pressurizing the preform, the metal powders are combined with each other by atom movement to form a structure, and a composite material including the water-soluble salt is formed thereinto. When the composite material is immersed into water, only the water-soluble salt is dissolved in water to prepare a metal porous structure having pores. Moreover, the metal porous structure may be prepared by completely melting the metal materials and introducing a foaming agent producing a gas thereto.

Meanwhile, in the case where the porous structure is prepared using an electrolyte including polymers and metal ions, the step a) is as follows.

First, the surface of a porous polymer is plated with the electrolyte including metal ions. In this case, the metal ions may include one or more selected from the group consisting of titanium (Ti) ions, cobalt (Co) ions, chromium (Co) ions and zirconium (Zr) ions, but are not limited thereto. Subsequently, the polymer is removed by increasing the temperature and, thus a porous metal structure can be prepared.

Meanwhile, in the case where the porous structure is prepared using a ceramic, the step a) is as follows.

First, ceramic fine powder and a binder (polymer) are mixed with each other. The mixture is applied on the surface of a framework of a removable foam material, such as polyurethane, and then dried to prepare a porous structure. Subsequently, when the porous structure is heated, the polymer is burned and removed near the combustion temperature of the binder polymer, and when the porous structure is further heated, residual ceramic powders are sintered, thereby preparing a porous structure having mechanical strength.

Here, the ceramic fine powder may be one or more selected from the group consisting of hydroxyapatite (HA) powder, zirconia powder and alumina powder.

In the step a), the method of preparing the above-mentioned porous structure may be modified or combined thereto. Further, the above porous structure may be prepared such that its inner and outer porosities are different from each other by applying to a part of different kinds of materials. In the latter, the inner portion of the porous structure has high density because it has no pores or low pores, and its outer portion has high porosity. Accordingly the porous structure can be prepared different porosity according to its positions. This porous structure can be used to manufacture an implant which can induce high bone formation rate on the surface thereof and can exhibit high resistance to external stress.

The above-mentioned methods of preparing a porous structure may be some examples of many various methods of preparing a porous structure, and the scope of the present invention is not limited thereto.

According to the method of manufacturing a biodegradable composite implant, in the step b), the pores of the porous structure can be impregnated with molten magnesium or molten magnesium-based alloy.

The magnesium-based alloy is melted as follows. Since magnesium is ignited at a very low temperature (of about 450° C., which changes according to which alloy elements are added), it is necessary to take a particular step at melting the magnesium. In a process of manufacturing a commercially available magnesium-based alloy, a very small amount (10 ppm or less) of beryllium (Be) is added to a molten magnesium-based alloy solution, and then the surface of the molten magnesium-based alloy solution is covered with a mixed gas of SF₆, CO₂ and dry air, so that a compact mixed film including MgNₓ, BeO, MgO, MgF₂, MgS and the like is formed on the surface of the solution, with the result that it is possible to prevent the molten magnesium-based alloy solution from reacting with oxygen, thereby allowing for operations to be conducted stably. However, in the case of biomaterials which require careful attention to prevention of introduction of impurities, since an oxide formation element, such as beryllium (Be), cannot be added to the magnesium-based alloy, it is preferred that the magnesium-based alloy be melted under a vacuum atmosphere or an inert gas atmosphere such as argon (Ar) which does not react with the magnesium alloy. The magnesium-based alloy can be melted using various methods, such as a resistance heating method generating heat by electrifying the resistor and resistant materials, an induction heating method flowing electric current into induction coil, a laser or a focused light method and the like. Among these methods, the resistance heating method is the most economical. Further, it is preferred that the molten magnesium-based alloy solution be stirred in order to uniformly mix the constituents thereof with each other at melting the magnesium-based alloy.

Methods of filling the pores of a porous structure with the magnesium-based alloy melted in this way may include a method of immersing the porous structure into the molten magnesium-based alloy solution, a method of fixing the porous structure and then flowing the molten magnesium-based alloy solution into the porous structure to fill the pores thereof, and a method of easily filling the pores of the porous structure with the molten magnesium by applying a pressure of 1 atm or more thereto in any of the above-described two methods. In this case, the molten magnesium can be easily filled in the pores of the porous structure by heating the porous structure such that the molten magnesium is not solidified while filling the pores therewith or by removing various pollutants from the surface of the porous structure.

Further, the step b) may be as follows. Specifically, first, magnesium or magnesium-based alloy is maintained and vaporized at a high temperature, preferably 700° C. or more, and then the vaporized magnesium or magnesium-based alloy passes through the pores of a porous structure and is thus deposited on the surface of the pores of the porous structure, thereby filling the pores of the porous structure with the magnesium or magnesium-based alloy.

Alternatively, the step b) may be as follows. Specifically, a salt including magnesium is dissolved in a liquid, and then a porous structure passes through the liquid, thereby causing the magnesium to be adsorbed on the pores of the porous structure.

In addition to the above-mentioned methods, according to a modified method thereof, the pores of the porous structure may be filled with magnesium-based alloy partially, not completely. That is, the porous structure is filled with molten magnesium-based alloy, and then high-pressure gas is blown onto the porous structure or the porous structure is rotated or shaken before the magnesium-based alloy is completely solidified. As the result, the non-solidified magnesium-based alloy is removed from the porous structure, and a part of the magnesium alloy remains in the pores of the porous structure, thereby preparing a composite material whose pores are partially impregnated with the magnesium. In this case, filling rates of the magnesium-based alloy may be controlled differently depending on regions of the pores of the porous structure.

According to another modified example thereof, it will be additionally expected that magnesium-based alloy is stuck only to the framework surface of the porous structure, and the pores of the porous structure are controlled to be partially left, and thus fine blood vessels necessary for forming bone can be easily formed in the inner portion of implant and simultaneously bone can be easily formed by the magnesium.

Meanwhile, in the case of a polymer having a melting point lower than that of magnesium-based alloy, when a porous structure is prepared at first and then the pores of the porous structure are filled with the molten magnesium-based alloy, the polymer porous structure cannot keep its shape. Accordingly, it is preferred that a biodegradable implant including a polymer and a magnesium-based alloy be manufactured by mixing magnesium-based alloy powder with the polymer in a volume ratio of 5:95˜95:5 and then heating the mixture to a temperature of 150˜500° C. and then pressurizing the heated mixture to a pressure of 1˜100 atms. Although the above conditions are preferred in a preparation of a polymer-magnesium (Mg) biodegradable implant, the polymer-magnesium (Mg) biodegradable implant may be formed under the other conditions. Therefore, the right of the present invention cannot be infringed by the change of the conditions for manufacturing the polymer-magnesium (Mg) biodegradable implant.

The above-described methods of preparing metals, ceramics and polymer porous structures, methods of filling the pores of the porous structure with magnesium-based alloy and methods of manufacturing the polymer-magnesium biodegradable implant are set forth to illustrate examples of the present invention, but the scope of the present invention is not limited thereto.

The step b) may further include the step of processing the magnesium-based alloy for controlling the biodegradation rate through one or more processes selected from the group consisting of a cooling process, an extrusion process, and a metal working process.

The cooling process can be used to improve the mechanical strength of magnesium-based alloy. Specifically, the cooling process can be used by immersing a crucible including the molten magnesium-based alloy into water. Further, the cooling process can be used by spraying the molten magnesium-based alloy with inert gas such as argon (Ar) and the like. In the method of cooling the magnesium-based alloy by spraying the molten magnesium alloy with inert gas, since the magnesium alloy is more rapidly cooled, and a very fine texture can be formed. However, when the magnesium-based alloy is molded in a small size, careful attention is required because a large number of pores (black part) may be formed therein.

The extrusion process is used to provide uniformity to the texture of magnesium-based alloy and to improve the mechanical properties thereof. The extrusion process may be conducted at a temperature of 300˜450° C. Further, the extrusion of the magnesium-based alloy may be conducted such that its extrusion ratio (the reduction ratio of cross section of the magnesium-based alloy before and after the extrusion of the magnesium-based alloy) is in the range of 10:1˜30:1. As the extrusion ratio is increased, the fine texture of the extruded material become uniform, and defects formed during the molding can be easily removed. However, in this case, it is required to increase the capacity of an extruding apparatus.

The metal working process may be used without any particular limitation as long as it is a metal working process well known to those skilled in the art. For example, the metal working process may include a process of directly molding magnesium-based alloy by pouring the above-mentioned molten magnesium-based alloy into a mold having a similar shape to that of the final product, a process of forming magnesium-based alloy into an intermediate material having a bar or plate shape or the like and then lathing or milling the intermediate material, and a process of forging magnesium-based alloy into a final product shape under high pressure, and the like.

### MODE FOR THE INVENTION

Hereinafter, methods of manufacturing a biodegradable implant including magnesium or a magnesium-based alloy for controlling biodegradation rate will be described in more detail with reference to the following Examples. However, the following Examples are set forth to illustrate the present invention, but the scope of the present invention is not limited thereto.

### Example 1

### Manufacture of an Alumina Implant Filled with Magnesium

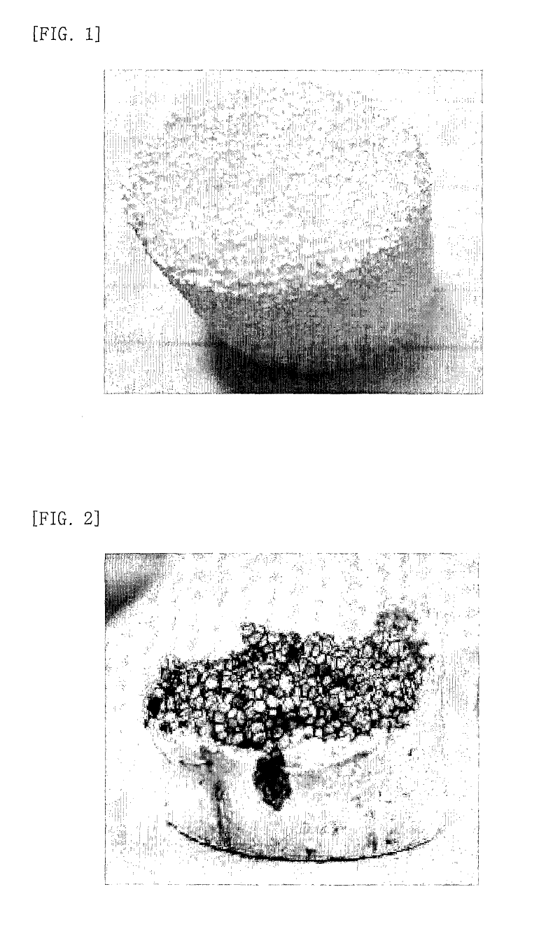

200 mL of ethanol, as a solvent, 6 g of polyvinyl butyral-co-vinyl alcohol-co-vinyl acetate (PVB), as a binder, 6 mL of triethyl phosphate (99.8%), as a dispersant, and 50 g of alumina, as a biodegradable ceramic, were mixed and then stirred for 2 hours to form a mixed solution. Subsequently, zirconia balls were put into the mixed solution and ball-milling was performed for about 24 hours. Then, polyurethane cut in a predetermined size and shape was immersed into the mixed solution, taken out, and then rotated at room temperature to prevent the pores from clogging by the solution. Subsequently, the polyurethane was dried in the air for about 5 minutes to cause the polyurethane foam material to be covered with highly-concentrated alumina. As described above, the process of immersing the polyurethane into the mixed solution and then drying the polyurethane was repeatedly conducted, thus forming an alumina mixed film having a thickness of 50˜1000 μm on a polyurethane framework. Subsequently, the dried foam material was further dried at about 60° C. for about 10 minutes in an oven, and then heat-treated. The heat treatment was conducted at a temperature of 800° C. (vaporization of polyurethane) for 3 hours due to a temperature increase of about 5° C. at a minute and then at a temperature of 1000˜1500° C. (sintering temperature depending on powders) for 3 hours in the same temperature increase condition to prepare an alumina porous structure having a diameter of 3 cm and a height of 4 cm. FIG. 1 shows the outer appearance of the prepared alumina porous structure.

The prepared alumina porous structure was impregnated with magnesium as follows. First, 1 kg of magnesium (manufacturing company: Timminco Metals, brand name: PURCH Magnesium ASTM 99.98% INGOT) was put into in a resistance heating furnace installed in a vacuum chamber. A metal mold with a passage having a diameter of 3 cm was installed under the heating furnace, and the prepared alumina porous structure was positioned in the inner passage. A vacuum atmosphere was formed such that the vacuum chamber had an inner pressure of 10⁻⁴ torr or less, and then high-purity argon of 99.99% or more was introduced into the vacuum chamber. Under a high-purity argon atmosphere, the molten magnesium solution was heated to a temperature of 700° C., and the metal mold was heated to a temperature of 500° C., and then a stopper was removed from the heating furnace, and thus the molten magnesium flowed into the alumina porous structure positioned in the metal mold, thereby manufacturing an alumina implant whose pores are filled with magnesium.

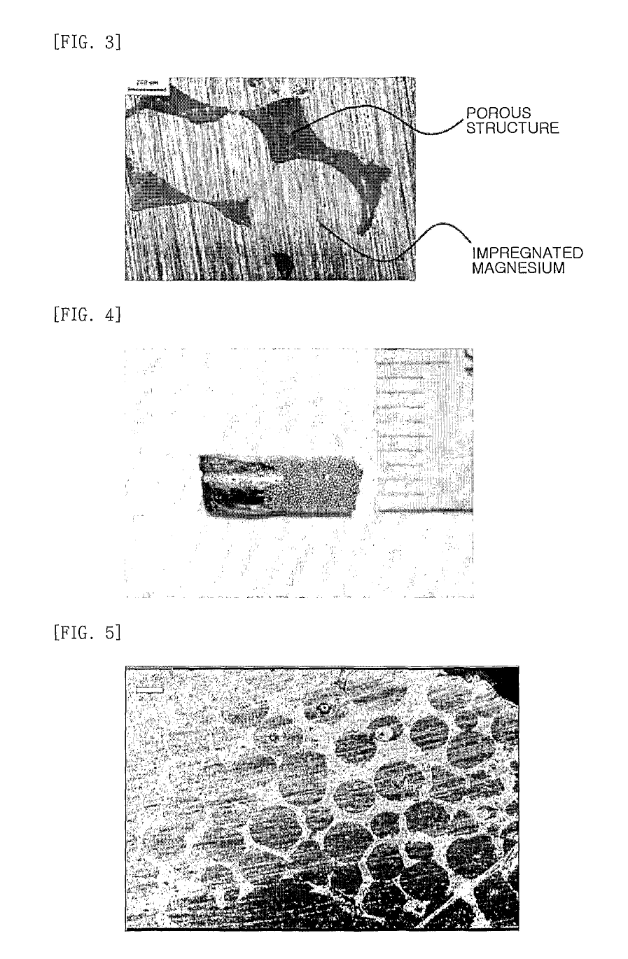

FIG. 2 shows the outer appearance of the alumina implant filled with magnesium manufactured as described above. Further, FIG. 3 is a photograph showing the enlarged section of an alumina implant filled with magnesium. In FIG. 3, the magnesium impregnated in the alumina implant is represented by gray parts, and the alumina porous structure is represented by relatively dark gray parts.

### Example 2

### Manufacture of a Titanium Implant Filled with Magnesium

The titanium porous structure was prepared by a rotating electrode method in which spherical titanium (Ti) powder having a diameter of 100˜200 μm was interposed between conductive electrodes and then an voltage which is charged in a 450 μF capacitor on condition of 1.0 kJ or 1.5 kJ is instantaneously discharged using a high-vacuum switch while current and voltage passing through the powder during the electric discharge are controlled, thus executing rapid sintering of the spherical titanium (Ti) powder.

A copper electrode bar was provided under a quartz tube having an inner diameter of 4.0 mm, and 0.7 g of sorted titanium powder was introduced into the quartz tube, and then the titanium powders were sufficiently packed each other using a vibrator. Meanwhile, 10 kg of a load was applied to the upper copper electrode bar using an automatic loading apparatus to connect the copper electrode to the upper part of titanium powder, and then a low-vacuum discharge was performed while maintaining the pressure in a discharge chamber at a vacuum of about 2×10⁻³ torr. The voltage and current passing through the titanium powder at the discharging were respectively measured in real time using a high-voltage probe and a high-current probe to control the structure of the porous structure.

FIG. 4 shows the outer appearance of a titanium porous structure used to manufacture a titanium implant filled with magnesium (Mg).

The titanium porous structure was impregnated with magnesium through the following processes. First, magnesium (manufacturing company: Timminco Metals, brand name: PURCH Magnesium ASTM 99.98% INGOT) was put into a crucible having an inner diameter of 50 mm, made of stainless steel (SUS 410). Subsequently, magnesium was melted by increasing the temperature of the crucible to a range of 700˜750° C. using a resistance heating furnace, while blowing argon (Ar) gas around the crucible such that magnesium is not brought into contact with air. The molten magnesium was stirred by shaking the crucible so as to obtain a sufficient mixing effect. Subsequently, a titanium porous structure, which was preheated to about 200° C., was immersed in the molten magnesium for about 5 minutes, taken out from the crucible and then water-cooled. FIG. 5 is a photograph showing the enlarged section of a titanium implant sample including the titanium porous structure, which was observed after it was taken out from the completely-cooled crucible, and a part of the porous structure was delivered and then abraded. In FIG. 5, the titanium porous structure is represented by dark gray parts, and the magnesium filled in the titanium implant is rep-resented by relatively light gray parts. As shown in FIG. 5, it can be seen that a implant material whose pores are filled with magnesium can be easily manufactured even through a method of immersing a titanium porous structure into a molten magnesium-based alloy.

### Test Example 1

### Measurement of the Strength of an Alumina Implant Impregnated with Magnesium

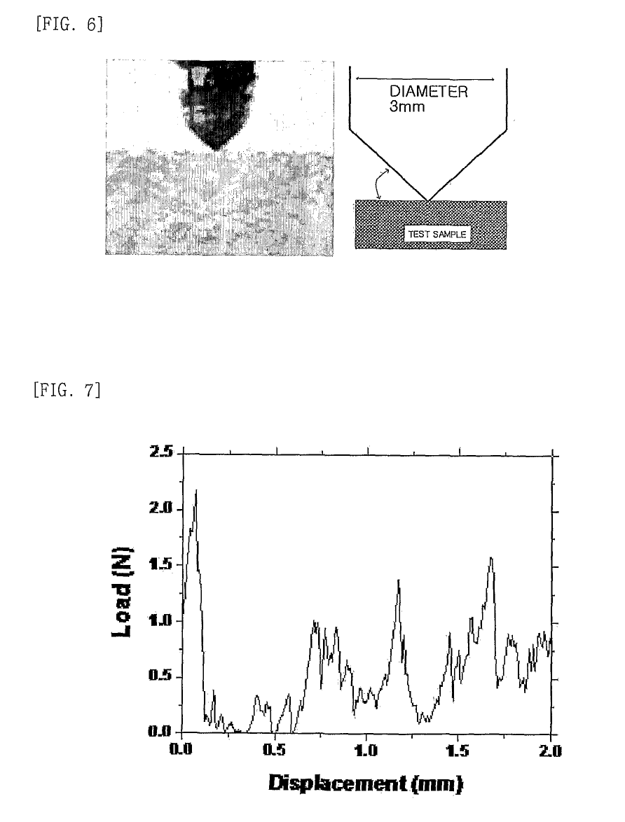

In order to evaluate the change in strength of an alumina implant filled with magnesium according to Example 1 of the present invention, the surface of the alumina material filled with magnesium, manufactured in Example 1, was abraded 100 times using emery paper and then disposed under a compression-tensile tester. Then, a tip having a diameter of 3 mm and an inclination of 45° was attached to a moving head of the compression-tensile tester, and then the tip descended at a velocity of 1 mm/min to press the surface of the alumina implant sample. In this case, the moving distance of the tip was limited to the maximum 2 mm. FIG. 6 shows the scene in which a tip for compression test presses the surface of the alumina foam prepared in the present invention and shows the schematic view of the tip.

FIG. 7 is a graph showing the results of measuring the compression strength of an alumina porous structure filled with no magnesium-based alloy. From FIG. 7, it can be seen that a force of a maximum of 2.2 N was applied to a test sample, and that when the tip for compression test moved forward, the porous structure was damaged, and thus the strength applied to the test sample was irregularly changed

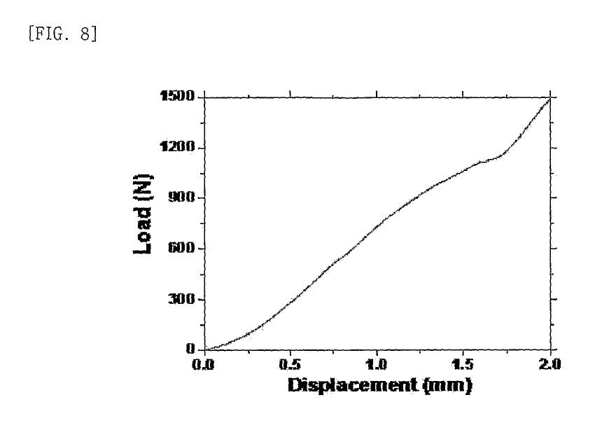

FIG. 8 is a graph showing the results of measuring the compression strength of an alumina implant filled with a magnesium-based alloy. The force applied to a test sample was increased to 1500 N as a tip for the compression test penetrated the surface of the test sample more deeply. The strength of this alumina implant was increased to approximately 680 times, compared to that of an alumina foam material whose pores are not filled with a magnesium-based alloy.

1. A composite implant comprising pores of a porous structure filled with a biodegradable magnesium-based alloy.

2. The composite implant according to claim 1, where in the biodegradable magnesium-based alloy is represented by Chemical Formula 1 below,
MgₐCa_{b}X_{c} <Chemical Formula 1>
wherein a, b and c are molar ratios of respective components and are in the range of 0.5≦a≦1, 0≦b≦0.4, and 0≦c≦0.4; and
X includes one or more selected from the group consisting of zirconium (Zr), molybdenum (Mo), niobium (Nb), tantalum (Ta), titanium (Ti), strontium (Sr), chromium (Cr), manganese (Mn), zinc (Zn), silicon (Si), phosphorus (P), nickel (Ni), iron (Fe) and selenium (Se).

3. The composite implant according to claim 1, wherein the porous structure is comprised of a metal selected from the group consisting of titanium, titanium alloys, cobalt-chromium alloys and stainless steels.

4. The composite implant according to claim 1, wherein the porous structure is comprised of a ceramic selected from the group consisting of calcium phosphate, alumina, zirconia and magnesia.

5. The composite implant according to claim 1, wherein the porous structure is comprised of a polymer selected from the group consisting of polyethylene, polylactic acid (PLA), polyglycolic acid (PGA), and copolymers (PLGA) thereof.

6. The composite implant according to claim 1, wherein the porous structure has a porosity of 5˜95%.

7. The composite implant according to claim 1, wherein the degradable magnesium-based alloy is filled in all pores of the porous structure or depending on regions of the pores of the porous structure such that the pores of the porous structure have different filling rate.

8. The composite implant according to claim 1, wherein the composite implant is an orthopedic implant, a dental implant, an implant for plastic surgery, or an implant for a blood vessel.

9. A method of manufacturing a composite implant, comprising the steps of:
a) preparing a porous structure; and
b) filling pores of the porous structure with a biodegradable magnesium-based alloy to form a composite material.

10. The method of manufacturing a composite implant according to claim 9, wherein the step b) further comprises one or more processes selected from the group consisting of heat treatment process, working process, and forming process.

11. The method of manufacturing a composite implant according to claim 9, wherein the biodegradable magnesium alloy is represented by Chemical Formula 1 below:
MgₐCa_{b}X_{c} <Chemical Formula 1>
wherein a, b and c are molar ratios of respective components and are in the range of 0.5≦a≦1, 0≦b≦0.4, and 0≦c≦0.4; and
X includes one or more selected from the group consisting of zirconium (Zr), molybdenum (Mo), niobium (Nb), tantalum (Ta), titanium (Ti), strontium (Sr), chromium (Cr), manganese (Mn), zinc (Zn), silicon (Si), phosphorus (P), nickel (Ni), iron (Fe) and selenium (Se).
